(19) United States
(12) Patent Application Publication
(10) Pub. No.: US 2006/0024235 A1
(43) Pub. Date: Feb. 2, 2006

(54) Stabilised superparamagnetic particles

(76) Inventor: Herbert Pilgrimm, Sophie-Charlotte (DE)

Correspondence Address: PENDORF & CUTLIFF, 5111 MEMORIAL HIGHWAY, TAMPA, FL, 33634-7356, US

(21) Appl. No.: 10/529,457

(22) PCT Filed: Oct. 9, 2002
(86) PCT No.: PCT/DE02/03862

§ 371 Date: Mar. 28, 2005

(51) Int. Cl. A61B 5/055

(52) U.S. Cl. 424/9.3

(57) ABSTRACT

The invention relates to stabilized superparamagnetic particles which consist of superparamagnetic single domain particles and aggregations of superparamagnetic particles which are stabilized with aliphatic dicarboxylic or polycarboxylic acids and which contain charged ions of chemical elements on the surface of the small superparamagnetic single-domain particles and, optionally, an additional tissue binding substance or pharmacologically effective substance. The superparamagnetic particles consist of a mixture of small superparamagnetic single domain particles have a particle size ranging from 3 to 5 namometers and stable, degradable aggregations of small superparamagnetic particles having a particle size of 10-1000 nanometers, and are made of iron hydroxide, iron oxide hydrate, iron oxide, iron mixed oxide or iron. The novel particles can be used as bacteriostatics and radio pharmaceuticals harming tumors, in order to prevent restenosis, in order to combat inflammatory diseases, for the functional control of organs, for magnetic drug targeting, as MR contrasting agents, as magnetic ion exchangers and magnetic adsorbients for separation methods, in the production of extremely small metal particles, as magnetic particles for in vitro diagnosis, optionally in conjunction with magnetic fields.

### BACKGROUND OF THE INVENTION

1. Field of the invention

The invention relates to superparamagnetic particles comprising superparamagnetic single domain particles and aggregates of superparamagnetic single domain particles of iron oxides or iron mixed oxides or iron which superparamagnetic particles are stabilised on their surface and can be used in medicine or medical diagnosis.

2. Related Art of the Invention

EP 0772776 B1 discloses superparamagnetic particles which superparamagnetic particles comprise superparamagnetic single domain particles and aggregates of superparamagnetic single domain particles on whose surfaces are bonded organic substances which organic substances occasionally comprise further bonding points for the coupling of tissue-specific bonding substances or diagnostically or pharmaceutically active substances. The superparamagnetic particles consist of a mixture of small superparamagnetic single domain particles having a particle size ranging between 3 and 50 Nanometers and stable degradable aggregates of small superparamagnetic single domain particles having a particle size ranging between 10 and 1000 nanometers and consisting of iron hydroxide, iron oxide hydrate, iron oxide, iron mixed oxide or iron which superparamagnetic single domain particles carry bonded to their surface aromatic substances containing monohydroxyl and/or polyhydroxyl groups, polyglycerines, substances containing amino acids, substances of orthosilicic acid and its condensation products containing silicate groups and substances of orthophosphoric or metaphosphoric acids and their condensation products containing phosphate groups which superparamagnetic single domain particles may comprise further bonding points.

EP 0888545 B1 discloses superparamagnetic single domain particles having increased R1 relaxivity and further having surface stabiliser substances whose particles comprise iron hydroxide, iron oxides, iron mixed oxides or iron and having a particle size ranging between 1 and 10 nanometers and further having an average particle diameter d₅₀ of 2 to 4 nanometers and further an increased R₁ relaxivity ranging between 2 to 50 and having a ratio of relaxivities R₂/R₁ of less than 5. To their surfaces are bonded low molecular stabiliser substances such as citric acid which low molecular stabiliser substances prevent an aggregation and sedimentation in gravity or in a magnetic field.

### SUMMARY OF THE INVENTION

The object of the invention is to extend the range of substances that can be bonded to the surface of the single domain particles in order to permit the optimum adapting of the physical, chemical and physiological characteristics of the resulting magnetic particles with respect to prevailing application areas wherein said substances should be stable and easy to manufacture.

The superparamagnetic particles described in EP 0772776 B1 comprising superparamagnetic single domain particles to whose surface are bonded organic substances which organic substances can also be stabilised by means of the low molecular aliphatic dicarbon and polycarbon acids described in EP 0888545 B1 such as malic acid, tartaric acid, citric acid, aspartic acid with respect to sedimentation in the earth's gravity or a magnetic field. The aggregates of superparamagnetic single domain particles described in EP 0772776 B1 can also be stabilised with respect to sedimentation in the earth's gravitational field e.g. by way of the low molecular citric acid described in EP 0888545 B1.

It has been discovered that stabilised superparamagnetic particles comprising superparamagnetic single domain particles of iron hydroxide, iron oxihydrate, iron oxide, iron mixed oxide or iron having a particle size ranging between 2 and 50 Nanometer

or aggregates thereof which aggregates comprise a particle size ranging between 10 and 1000 nanometers or mixtures thereof which particles or aggregates are respectively stabilised on their surface by means of aliphatic dicarbon or polycarbon acids or derivatives thereof can carry charged ions bonded to their surface. The ions form very stable bonds with the surface of the superparamagnetic particles which bonds do not influence the sedimentation stability of the superparamagnetic single domain particles and aggregates in predetermined concentration ranges.

The stability characteristics of the dispersions containing metal ions have been investigated up to a portion of metal ions of up to 10% mol of the ion portion of the magnetic particles. It was found that in all the investigated cation types the stability of the dispersions was not changed up to a metal ion portion of 5% mol.of the iron portion of the magnetic particles. Surprisingly in the case of all samples the ion concentrations of the added metal ions in the ultrafiltrate of the dispersions lay below the respective proof limit of the measuring method which ion concentrations were measured by means of atom absorption spectroscopy (AAS). Only above a metal ion portion of 5% mol of the iron portion of the magnetic particles does the stability of the dispersion reduce depending upon the type of element while the portion of added metal ions and the ion concentration measured in the ultrafiltrate of the dispersions lay within the measurement range of the AAS.

Preferred ions of charged chemical elements are positively charged metal ions selected from the group comprising metal ions of the chemical elements copper, silver, gold, iron, nickel, cobalt, gallium, thallium, bismuth, palladium, rhenium, rhodium, ruthenium, platinum, technetium, indium, iridium, osmium, radium, selenium, vanadium, yttrium, zircon, rare earths, mixtures thereof and radioactive isotopes of said elements.

In a further embodiment of the invention the metal ions are selected from the group of radioactive isotopes, comprising ⁵²Fe, ⁶⁷Ga, ⁹⁹Tc, ¹¹³In, ¹⁸⁸Rh, ¹⁹²Ir, ¹⁹⁸Au, ²⁰¹Tl and ²²³Ra.

A preferred group of positively charged metal ions are selected from the group comprising metal ions of the chemical elements copper, silver, gold, platinum, palladium, osmium, rhenium, rhodium, ruthenium, vanadium and mixtures thereof.

In a further embodiment of the invention the charged ions are non-metal ions that are bonded by means of a polyethylenimine bridge to the surface of the superparamagnetic single domain particles. Preferably the radioactive isotopes ¹³N, ¹⁵O, ¹⁸F, ¹²³J or compounds thereof are bonded by means of said polyethylenimine bridge to the stabilised superparamagnetic particles.

Along with the charged ions of chemical elements as a further beneficial embodiment of the invention there are occasionally bonded tissue-specific bonding substances to the surface of the superparamagnetic particles. These substances can be selected from the group comprising antigenes, antibodies, ribonucleic acids, deoxyribonucleic acids, ribonucleic acid sequences, deoxyribonucleic acid sequences, haptene, avidin, streptavidin, protein A, protein G, endotoxin-bonding proteins, lectins, selectins, surface proteins of organelles, viruses, microbes, algae, fungi.

Along with the charged ions of chemical elements as a further beneficial embodiment of the invention occasionally pharmaceutically active substances can be bonded to the surfaces of the superparamagnetic particles which pharmaceutically active substances are selected from the group comprising antitumor proteins, enzymes, antitumor enzymes, antibiotics, plant alkaloids, alkylation reagents, antimetabolites, hormones and hormone antagonists, interleukines, interferones, growth factors, tumor necrosis faktors, endotoxins, lymphotoxins, urokinases, streptokinases, plasminogen streptokinase activator complex, tissue plasminogen activators, desmodus plasminogen activators, macrophagic activating bodies, antisera, blood and cell components and their decomposition products and derivatives, cell wall components of organelles, viruses, microbes, algae, fungi and their decomposition products and derivatives, protease inhibitors, alkylphosphocholine, substances containing radioactive isotopes, surfactents, cardiovascular pharmaceutical agents, chemotherapeutic agents, gastrointestinal pharmaceutical agents and neuropharmaceutical agents.

“Derivatives of aliphatic dicarbon or polycarbon acids” refers particularly to monofunctional esters in the case of dicarbon acids or monofunctional or difunctional esters in the case of polycarbon acids containing C₁-C₁₈ alkyl portions and preferably C₁-C₄ alkyl portions.

The manufacture of the superparamagnetic particles is carried out according to the prior art by means of a precipitation of an iron salt solution by means of e.g. ammoniac water and a subsequent targeted agglomeration of the resulting superparamagnetic single domain particles. In this case the superparamagnetic single domain particles are stirred into water and brought to a state of aggregation at a pH value of 1 to 7 by heating to 80 to 120° C. and at temperatures over 100° C. in the autoclave. After the cooling of the dispersion the particles are washed until the electrical conductivity of the filtrate is less than 10 μS/cm. The superparamagnetic particles manufactured in said prior art manner immediately form a rapidly precipitating sediment that cannot be reduced to a stable dispersion even when stirred vigorously or subjected to ultrasound treatment. Only the bonding of stabiliser substances to the surface of the superparamagnetic particles enables them to disperse. In the case of citric acid as the stabiliser substance, stirring with the glass rod is sufficient while in the case of other stabiliser substances a greater input of energy is required e.g. heating or the effect of ultrasound, in order to obtain stable dispersions.

After the stabilising of the superparamagnetic particles with an aliphatic dicarbon or polycarbon acid e.g. with citric acid the pH value of the dispersions are adjusted to 7.0 with bases such as caustic soda or methylglucamine and dialysed with water or physiological salt solution in order to remove the excess portion of electrolyte.

In accordance with the invention the dispersion of superparamagnetic particles which dispersion of superparamagnetic particles can contain an iron portion ranging from 0.001 mol Fe/l to 10 mol Fe/l and further can disperse in water or a low boiling-point organic polar solvent is now mixed with an aqueous solution of ions of chemical elements. The applicable concentration range of the solutions of the ions of chemical elements ranges from 0.001 mmolar to 1 molar. The proportion of ions of chemical elements with respect to iron in the mixture should not exceed 10% mol.

It is beneficial to use dilute solutions, e.g. between 0.001 and 0.1 molar solutions, and to add said solutions slowly e.g. drop by drop in order to avoid a large localised concentration gradient.

The ions of chemical elements such as the positively charged metal ions of the chemical elements copper, silver, gold, iron, nickel, cobalt, gallium, thallium, bismuth, palladium, rhenium, rhodium, ruthenium, platinum, technetium, indium, iridium, osmium, radium, selenium, vanadium, yttrium, zirconium and rare earths and mixtures thereof or of radioactive isotopes of said metal ions such as ⁵²Fe, ⁶⁷Ga, ^{99m}Tc, ¹¹³in, ¹⁸⁸Rh, ¹⁹²Ir, ¹⁹⁸Au, ²⁰¹Tl or ²²³Ra are preferably dissolved in water before mixing with the superparamagnetic particles in water or a low boiling-point organic polar solvent.

The negatively charged ions of chemical elements like the radioactive isotopes ¹³N, ¹⁵O, ¹⁸F, ¹²J are dissolved in an aqueous polyethylenimine solution before mixing with the superparamagnetic particles in water. The applicable concentration range of the polyethylenimine solution ranges from 0.001 to 1 molar and the applicable concentration range of the solutions of the negatively charged ions of chemical elements ranges from 0.001 mmolar to 1 mmolar.

The mixing of the charged ions of chemical elements with the superparamagnetic particles is carried out by stirring wherein it is important that the aqueous dispersion of the superparamagnetic particles is present and the aqueous solution of ions of chemical elements is added gradually e.g. drop by drop. The mixing takes place within a temperature range of 5° C. to 70° C. and preferably at room temperature i.e. at 20-25° C.

The stabilised superparamagnetic particle dispersion contains no or only weakly aggregated superparamagnetic single domain particles. These form a stable magnetic liquid which stable magnetic liquid can be separated easily from the larger superparamagnetic aggregates by means of their sedimentation in a magnetic field of corresponding strength and inhomogenity

In a simple execution of the magnetic separation one places a glass beaker containing the magnetic dispersion on a permanent magnet having a magnetic flux density of 0.1 T and pours off the remaining magnetic liquid after a sedimentation period of approx. 30 min. Remaining in the sediment are the superparamagnetic aggregates which superparamagnetic aggregates depending on particle size neither disperse again spontaneously in the dispersion nor remain as sediment. Up to particle sizes of approx 500 nm the superparamagnetic aggregates disperse neither spontaneously nor through gentle stirring in the aqueous dispersion agent.

For the method according to the invention it has been found that polyethylenimines form stable bonds on the e.g. with citric acid stabilised surface of the superparamagnetic particles which stable bonds do not affect the sedimentation stability of the superparamagnetic single domain particles or superparamagnetic aggregates in specific concentration ranges. With said polyethylenimine-coated magnetic particles also radioactive non-metal ions can bond to the surface of the superparamagnetic particles. The above cited short-lived radiopharmaceutical agents such as ¹³N, ¹⁵O, ¹⁸F, ¹²³J can then be bonded to the free amine groups of the polyamine compounds.

It has also been found that polyethylenimines also form stable bonds on the e.g. with citric acid-stabilised surfaces of the superparamagnetic particles which stable bonds do not affect the sedimentation stability of the superparamagnetic single domain particles and superparamagnetic aggregates in predetermined concentration ranges provided that the polyethylenimines are mixed beforehand with the short-lived radiopharmaceutical agents such as ¹³N, ¹⁵O, ¹⁸F, ¹²³J and are only then bonded to the surfaces of the superparamagnetic particles.

The stabilised superparamagnetic particles can be used as bacteriostatics or radiopharmaceutical agents for the purpose of tumour destruction, for the prevention of restenosis, for the combating of inflammatory diseases, for the control of organ functions, for magnetic drug targeting, as MR contrast agents, as magnetic ion exchangers and magnetic adsorbents for separation procedures and further as magnetic particles for in vitro diagnosis occasionally under the action of magnetic fields.

The superparamagnetic particles according to the invention which superparamagnetic particles contain ions and preferably metal ions can be used e.g. as bacteriostatics. Superparamagnetic particles to whose surface are bonded silver ions thus act as strong bactericides. Single domain particles or aggregates thereof containing silver can be used therefore as therapeutic agents e.g. in the case of inflammatory diseases of the stomach intestinal tract. The superparamagnetic particles containing silver are adsorbed at the bacterial inflammation focus and the bacteria oxygen supply is suppressed by the action of the small portion of silver ions with the result that the bacteria are killed.

Investigations on rats have shown that silver containing superparamagnetic single domain particles and aggregates such as example 3 can be used as an oral therapy for the treatment of inflammatory stomach intestine diseases and diseases caused by the bacteria type Helicobacter pylori.

Investigations on rats have shown that very small superparamagnetic single domain particles containing silver such as example 4 can also be used as a parenteral therapy in the case of bacterial inflammation processes in the body. The toxicity of the sample having a LD 50 of 3 mmol iron/kg body weight was suitable for therapeutic uses. In the case of a reduction of the silver ion concentration a reduction of the toxicity is to be expected

A benefit of said strongly bactericidal and single domain particles or aggregates thereof containing silver is that with the aid of nuclear spin tomography it is possible to diagnose the adsorption type and the adsorbed quantity of the magnetic particles.

Radioactive superparamagnetic particles can serve for the manufacture of a parenteral radiopharmaceutical agent for use both for the diagnosis and therapy of vulnerable plaques as well as for the diagnosis and therapy of restenosis after balloon angioplasty or stent implantation. By means of the T1 and T2 effects of the very small superparamagnetic single domain particle in accordance with EP 0888545 B1 (increased R₁-relaxivity ranging from 2 to 50 and a ratio of the relaxivities R₂/R₁ of less than 5) which ratio is likewise recorded here it is possible to investigate the concentration of the particles in the vessel walls with the aid of nuclear spin tomography. The therapeutic action of the radioactive superparamagnetic particles for the diagnosing and therapy of vulnerable plaques and for preventing restenosis after balloon angioplasty or stent implantation lies in the destruction of the cells responsible for re-growth in the plaques on the vessel walls. After the removal of plaques and after balloon angioplasty or stent implantation the parenteral radiopharmaceutical agent is injected directly via a hollow needle into the investigated area of the vessel in order to prevent restenosis by destroying the vessel walls responsible for the plaque formation.

Radioactive superparamagnetic particles having tissue-specific antibodies can be used as radiopharmaceutical agents for combating specific tumour types since after parenteral injection of the particles the tissue-specific antibodies dock onto the corresponding receptors of the tumour cells and the radioactive components of the magnetic particles destroy the tumour cells.

Diagnosis and therapy of glioblastomes with radioactive citrate-coated small superparamagnetic single domain particles is thereby possible.

The superparamagnetic particles can also be used for in vitro diagnosis or as magnetic ion exchangers and magnetic adsorbents for the separation of ions, organic molecules, macromolecules, cells, viruses etc. in bioengineering, wastewater purification or other substance separation methods providing that the corresponding ion exchanger groups and adsorbents are bonded to the surface of the particles. Superparamagnetic particles containing metal ions can also be used to manufacture extremely small metal particles wherein the iron oxide particles are dissolved in the presence of reductive substances by means of dissolved acid. The manufacture of catalysers with large surfaces is likewise possible.

The manufacture and characteristics of the superparamagnetic particles according to the invention are described with reference to examples.

### EXAMPLE 1

Iron (III) chloride (270 g) and iron(II) sulphate (153 g) are dissolved in 1 l distilled water. By stirring in caustic soda the pH-value is adjusted to 9.5. After successful precipitation the pH-value of the dispersion is adjusted by stirring in hydrochloric acid to 5.0 and heated to 100° C. After the cooling of the dispersion the sediment is washed until the filtrate displays an electrical conductivity of <10 μS/cm. The superparamagnetic particles are stabilised by mixing the particles with an aqueous solution of 120 g citric acid at room temperature. The pH-value of the dispersion is adjusted to 7.0 by adding caustic soda and the unbound salts are dialysed with distilled water until the electrical conductivity of the dialysate is <10 μS/cm. To remove larger or weakly aggregated superparamagnetic particles the dispersion is centrifuged at 10,000 rpm for 10 min and the centrifugate is concentrated by means of ultrafiltration with a 40 kD-filter to an iron portion of approx. 2 mol/l.

The superparamagnetic single domain particles comprise an average particle diameter of approx. 16 nm. The superparamagnetic particle aggregates that are situated in the sediment of the centrifuge comprise an average particle diameter of approx. 100 nm.

Typical analysis data of the very small superparamagnetic single domain particles is:

| particle diameter d50 | 8 nm |
|---|---|
| overall diameter | 16 nm |
| with stabiliser: | |
| iron (II) portion | 16% |
| T1 relaxivity | 12 l/mmol s |
| T2 relaxivity | 25 l/mmol s |
| Ratio of the relaxivities R2/R1 | 2.05 |

### EXAMPLE 2

Iron(III) chloride (270 g) and iron(II) chloride(119 g) are dissolved in 1 1 distilled water. The pH-value of the solution is adjusted to 9.6 by stirring in ammoniac water. After successfully sedimentation the dispersion is stirred for 10 minutes and displaced with a solution of 120 g citric acid in 500 ml water and further stirred for 10 min. After the cooling of the dispersion the sediment is washed until the filtrate displays an electrical conductivity of <10 μS/cm. The solid is stirred in 300 ml water and dispersed for 10 min by means of ultrasound at 100 W power. The resulting dispersion is sedimented for 30 min on a permanent magnet having a magnetic flux density of 0.1 T and the excess magnetic fluid poured off. The excess contains predominantly stabilised superparamagnetic single domain particles. The sediment on the permanent magnet contains the superparamagnetic degradable aggregates. The pH-value of the dispersion adjusted to 7.0 and the unbound salts with a physiological table salt until the dialysate comprises an ammonium portion of <0.001 g/l. To remove larger or weakly aggregated superparamagnetic particles the dispersion is centrifuged at 10,000 rpm for 10 min and the centrifugate is concentrated by means of ultrafiltration with a 40 kD-filter to an iron portion of approx. 2 mol/l.

The superparamagnetic single domain particles comprise an average particle diameter of approx. 14 nm. The superparamagnetic particle aggregates located in the sediment of the centrifuge comprise an average particle diameter of approx. 80 nm.

Typical analysis data of the very small superparamagnetic single domain particles is:

| particle diameter d50 | 4 nm |
|---|---|
| overall diameter | 8 nm |
| with stabiliser: | |
| iron (II) portion | 14% |
| T1 relaxivity | 19 l/mmol s |
| T2 relaxivity | 36 l/mmol s |
| Ratio of the relaxivities R2/R1 | 1.89 |

### EXAMPLE 3

To 20 ml of the superparamagnetic aggregates of example 1 having an iron portion of 2 mol/l are stirred in drop-by-drop 2 ml of a 0.1 molar silver nitrate solution at 25° C. until mixed in. The excess electrolyte solution is dialysed by means of dialysis with a 40 kD filter with distilled water until the electrical conductivity of the dialysate is <10 μS/cm. The resulting dispersion is sedimentation-stable and can be used according to corresponding pharmaceutical formulation as a bacteriostatic in the case of bacterial diseases of the stomach intestinal tract. The adsorption of the superparamagnetic aggregates in the stomach intestinal tract can be observed with the aid of nuclear spin tomography.

### EXAMPLE 4

To 20 ml of the small superparamagnetic single domain particles from example 1 having an iron portion of 2 mol/l, are stirred in drop-by-drop 2 ml of a 0.1 molar silver nitrate solution at 20° C. The excess electrolyte solution is dialysed by means of dialysis with a 40 kD filter with distilled water until the electrical conductivity of the dialysate is <10 μS/cm. The resulting dispersion is stable with respect to sedimentation and magnetic fields and can be used for the manufacture of a parenteral therapy in the case of bacterial inflammation processes in the body. The adsorption of the superparamagnetic aggregates in the stomach intestinal tract can be observed with the aid of nuclear spin tomography.

### EXAMPLE 5

20 ml of the small superparamagnetic single domain particles from example 2 having an iron portion of 2 mol/l are displaced with 2 ml of a radioactive gallium 67 citrate solution having an activity of 400 MBq (Mega Becquerel) and an effective dose of 48 SV (Sievert). The superparamagnetic single domain particles comprise an average particle diameter of approx. 14 nm. The resulting dispersion is stable with respect to sedimentation and magnetic fields and can be used for the manufacture of a parenteral radiopharmaceutical agent for use for the diagnosis and therapy of vulnerable plaques and of restenosis after balloon angioplasty or stent implantation. By means of the T1 and T2 effects of the very small superparamagnetic single domain particles there is obtainable a concentrating of the particles in the vessel walls with the aid of nuclear spin tomography.

A diagnosis and therapy of glioblastomes is likewise possible with said radioactive citrate-coated small superparamagnetic single domain particles.

### EXAMPLE 6

20 ml of the small superparamagnetic single domain particles from example 2 having an iron portion of 2 mol/l are displaced with 2 ml of a radioactive gallium 67 citrate solution having an activity of 400 MBq (Mega Becquerel) and an effective dose of 48 SV. The superparamagnetic aggregates comprise an average particle diameter of approx. 80 nm. The resulting dispersion is stable with respect to sedimentation and can serve for the manufacture of a parenteral radiopharmaceutical agent. The superparamagnetic aggregates from example 2 can be used for diagnosis and therapy of malign liver tumours within the context of locoregional radiotherapy (radio embolisation).

### EXAMPLE 7

20 ml of the small superparamagnetic single domain particles from example 2 having an iron portion of 1 mol/l, are displaced with 4 ml of a 0.1 millimolar pentaethylenehexamine solution. To said dispersion are added radioactive jodide 123-solution having an activity of 300 MBq and an effective dose of 2.3 SV. The superparamagnetic single domain particles comprise an average particle diameter of approx. 14 nm. The resulting dispersion is stable with respect to sedimentation and magnet fields and can serve for the manufacture of a parenteral radiopharmaceutical agent.

The free amine groups of pentaethylenehexamine are used for the coupling of tissue-specific bonding substances such as antibodies of CD 30 receptors of Hodgkins lymphoma or antibodies of GD2 receptors of neuroblastomers.

### EXAMPLE 8

20 ml of the small superparamagnetic single domain particles from example 2 having an iron portion of 2 Mol/l are displaced with 2 ml of a 0.1 molar platinum II-chloride solution which molar platinum II-chloride solution is stirred in drop by drop at 20° C. The excess electrolyte solution is dialysed by means of dialysis with a 40 kD filter with distilled water until the electrical conductivity of the dialysate is <10 μS/cm. The superparamagnetic single domain particles comprise an average particle diameter of approx. 10 nm. The resulting dispersion is stable with respect to sedimentation and magnet fields and can serve for the manufacture of a platinum containing catalyser.

### EXAMPLE 9

20 ml of the small superparamagnetic single domain particles from example 2 having an iron portion of 2 mol/l are displaced with 1.5 ml of a mixture of 1 ml 0.1 molar platinum II chloride solution and 0.5 ml 0.1 molar rhenium III chloride solution which 1 ml 0.1 molar platinum II chloride solution and 0.5 ml 0.1 molar rhenium III chloride solution are stirred in drop by drop at 20° C. The excess electrolyte solution is dialysed by means of dialysis with a 40 kD filter with distilled water until the electrical conductivity of the dialysate is <10 μS/cm. The superparamagnetic single domain particles comprise an average particle diameter of approx. 10 nm. The resulting dispersion is stable with respect to sedimentation and magnet fields and can serve for the manufacture of a catalyser containing platinum and rhenium.

### EXAMPLE 10

The superparmagnetic single domain particles from example 8 are displaced with 1 molar oxalic acid solution and heated to 70° C. in order to dissolve the iron oxide particles. The yellow solution contains the very small nonometer-sized platinum particles. The excess electrolyte solution is dialysed by means of dialysis with a 3 kD filter with distilled water until the electrical conductivity of the dialysate is <10 μS/cm. The resulting dispersion of platinum particles is stable with respect to sedimentation and magnetic fields and can serve for the manufacture of a catalyser containing platinum.

1. Stabilised superparamagnetic particles comprising superparamagnetic single domain particles of iron hydroxide or iron oxihydrate or iron oxides or iron mixed oxide or iron having a particle size ranging between 2 and 50 nanometers, or aggregates thereof having a particle size ranging between 10 and 1000 nanometers, or mixtures thereof, respectively stabilised on their surface by means of aliphatic dicarbon or polycarbon acids or derivatives thereof, which stabilised acids or derivatives prevent an aggregation and sedimentation in gravity, wherein the superparamagnetic single domain particles carry charged ions of chemical elements bonded to their surface.

2. The particles according to claim 1 wherein the ions are positively charged metal ions selected from the group consisting of ions of the chemical elements copper, silver, gold, iron, nickel, cobalt, gallium, thallium, bismuth, palladium, rhenium, rhodium, ruthenium, platinum, technetium, indium, iridium, osmium, radium, selenium, vanadium, yttrium, zirconium, rare earths, mixtures of said positively charged metal ions and radioactive isotopes of said elements.

3. The particles according to claim 2 wherein the metal ions are selected from the group of radioactive isotopes consisting of ⁵²Fe, ⁶⁷Ga, ^{99m}Tc, ¹¹³in, ¹⁸⁸Rh, ¹⁹²Ir, ¹⁹⁸Au, ²⁰¹Tl and ²²³Ra.

4. The particles according to claim 2 wherein the positively charged metal ions are selected from the group consisting of metal ions of the chemical elements copper, silver, gold, platinum, palladium, osmium, rhenium, rhodium, ruthenium, vanadium and mixtures of said metal ions.

5. The particles according to claim 1 wherein the charged ions are non-metal ions which non-metal ions are bonded by means of a polyethylenimine bridge to the surface of the superparamagnetic single domain particles.

6. The particles according to claim 5, wherein the charged ions are those of the radioactive isotopes ¹³N, ¹⁵O, ¹⁸F, ¹²³J or mixtures of said radioactive isotopes.

7. The particles according to claim 1 wherein the superparamagnetic single domain particles are stabilised on their surface by means of malic acid, tartaric acid, citric acid, aspartic acid or mixtures thereof.

8. The particles according to claim 1 wherein the superparamagnetic single domain particles and the particles of the stable and degradable aggregates comprise iron, iron hydroxide, iron oxihydrate, γ-Fe₂O₃, Fe₃O₄, the iron mixed oxides of the general formula mMO.nFe₂O₃ wherein M refers to the bivalent metal ions Fe, Co, Ni, Mn, Be, Mg, Ca, Ba, Sr, Cu, Zn, Pt or mixtures of said bivalent metal ions or comprise the mixed oxides of the general formula mFe₂O₃.nMe₂O₃ wherein Me refers to the trivalent metal ions Al, Cr, Bi, rare earths or mixtures thereof wherein m and n are whole numbers ranging from 1 to 6.

9. The particles according to claim 1 wherein the superparamagnetic single domain particles comprise on their surface in addition to the stabilising carbon acids and the positively charged ions of chemical elements a tissue-specific bonding substance or a pharmacologically active substance or a mixture of said tissue-specific bonding substance or a pharmacologically active substance.

10. The particles according to claim 1 wherein the R₁-relaxivity of the superparamagnetic single domain particles lies in the range from 2 to 50 and the ratio of the relaxivities R₂/R₁ is less than 5.

11. A method for the manufacture of stabilised superparamagnetic particles comprising superparamagnetic single domain particles of iron hydroxide or iron oxihydrate or iron oxides or iron mixed oxide or iron having a particle size ranging between 2 and 50 nanometers, or aggregates thereof having a particle size ranging between 10 and 1000 nanometers, or mixtures thereof, respectively stabilised on their surface by means of aliphatic dicarbon or polycarbon acids or derivatives thereof, which stabilised acids or derivatives prevent an aggregation and sedimentation in gravity, from carbon acid-stabilised single domain particles or their aggregates which comprises
mixing the stabilised superparamagnetic single domain particles and aggregates or mixtures thereof with solutions containing ions of chemical elements wherein the concentration of the solutions lies in the range from 0.001 millimolar to 1 molar and wherein further the ratio of ions of chemical elements to iron is <10 mol-% and wherein the temperature is 5 to 70° C. and
subsequently ridding the particle dispersion of excess ions.

12. The method according to claim 11 wherein for the manufacture of stabilised particles with non-metal ions before mixing with the superparamagnetic particles the solutions having the non-metal ions are brought into contact with a polyethylenimine or the superparamagnetic particles treated with polyethylenimine are brought into contact with solutions that contain non-metal ions.

13. A pharmacologically active preparation comprising a pharmacologically acceptable carrier and superparamagnetic single domain particles of iron hydroxide or iron oxihydrate or iron oxides or iron mixed oxide or iron having a particle size ranging between 2 and 50 nanometers, or aggregates thereof having a particle size ranging between 10 and 1000 nanometers, or mixtures thereof, respectively to which particles or aggregates are bonded stabilising aliphatic dicarbon or polycarbon acids or derivatives thereof, which stabilising aliphatic dicarbon or polycarbon acids or derivatives prevent an aggregating and sedimenting in gravity and which additionally carry positively charged ions of chemical elements bonded to their surface.

14. The preparation according to claim 13 wherein the single domain particles of the aggregates comprise coupled to the stabilising carbon acid(s) in addition to the stabilising carbon acid and the metal ions a tissue-specific bonding substance or a pharmacologically active substance or a mixture of said tissue-specific bonding substance or a pharmacologically active substance.

15. A method for tumour destruction for the prevention of restenosis, for the combating of inflammatory diseases, for the control of organ functions, for the purpose of magnetic drug targeting, or as MR contrast agents, as magnetic ion exchangers and magnetic adsorbents for separation procedures, or for in vitro diagnosis using extremely small metal particles as magnetic particles optionally under the action of magnetic fields, said method comprising administering to a mammal in need thereof a bacteriostatic or radiopharmaceutical amount of stabilised superparamagnetic particles comprising superparamagnetic single domain particles of iron hydroxide or iron oxihydrate or iron oxides or iron mixed oxide or iron having a particle size ranging between 2 and 50 nanometers, or aggregates thereof having a particle size ranging between 10 and 1000 nanometers, or mixtures thereof, respectively stabilised on their surface by means of aliphatic dicarbon or polycarbon acids or derivatives thereof, which stabilised acids or derivatives prevent an aggregation and sedimentation in gravity, and wherein the superparamagnetic single domain particles carry charged ions of chemical elements bonded to their surface.
